(19) United States
(12) Patent Application Publication
(10) Pub. No.: US 2025/0367065 A1
(43) Pub. Date: Dec. 4, 2025

(54) TONGUE DEPRESSION AUXILIARY DEVICE

(71) Applicant: YU-SHIANG WU, Taichung City (TW)

(72) Inventors: YU-SHIANG WU, Taichung City (TW); CHU-WEI HUANG, Taichung City (TW)

(73) Assignee: YU-SHIANG WU, Taichung City (TW)

(21) Appl. No.: 19/304,018

(22) Filed: Aug. 19, 2025

Related U.S. Application Data
(63) Continuation-in-part of application No. 18/151,120, filed on Jan. 6, 2023.

(51) Int. Cl. A61H 21/00; A61B 13/00; A61F 5/56; A61H 7/00

(52) U.S. Cl. CPC A61H 21/00; A61B 13/00; A61F 5/566; A61H 7/001; A61H 2201/0192; A61H 2201/1604; A61H 2201/165; A61H 2201/1695; A61H 2205/022

(57) ABSTRACT

A tongue depression auxiliary device with a brace and a neuro-modulating member is disclosed. The brace is configured to be detachably positioned within a user's oral cavity. The neuro-modulating member is connected to the brace and extends therefrom toward a lower jaw, wherein a bottom side of the neuro-modulating member comprises at least one protrusion configured to exert a concentrated pressure on a specific functional region of the tongue, and the concentrated pressure is configured to sufficiently stimulate a deep myofascial layer of the tongue that contributes to an activation of a deep core muscle, wherein the concentrated pressure is proportional to a length of the protrusion of the neuro-modulating member protruded from the bottom side of the brace.

### CROSS-REFERENCE TO RELATED APPLICATION

This application claims the benefit of priority from, and is a continuation-in-part application of U.S. patent application Ser. No. 18/151,120, filed on Jan. 6, 2023, which is hereby incorporated herein by reference in its entirety.

### BACKGROUND OF THE INVENTION

### 1. Field of Invention

The invention relates to an auxiliary device for use in an oral cavity, and more particularly to a tongue depression auxiliary device configured to activate a deep core muscle by stimulating a deep myofascial layer of a tongue.

### 2. Related Art

Generally speaking, when exercising, the muscle tissue of the human body will use adenosine triphosphate (ATP) to produce repeated contractions. As the exercise time increases, the concentration of ATP will gradually decrease, resulting in decreased muscle function and muscle fatigue. In particular, it is more likely to cause dizziness, spiritless in many people when they are doing vigorous exercise. In severe cases, it can also cause chest tightness, shortness of breath, and asthma. At present, although there are techniques to promote aerobic exercise to train lung capacity, chest expansion exercise (such as stretching) to expand thoracic cavity to train lung capacity, or adjust breathing methods to specially use abdominal breathing to train lung capacity; however, these exercises or training must be carried out consciously before they can be completed.

The deep core muscles of the human body, including the diaphragm, transversus abdominis, and pelvic floor, are critical for postural stability, efficient breathing, and overall athletic performance. Conventionally, methods for strengthening these muscles involve conscious physical exercises, such as those taught in physical therapy or Pilates. These techniques often require a user's focused concentration and proprioceptive awareness to properly engage the target muscles.

However, the effectiveness of these conventional methods can be limited, as many individuals find it difficult to correctly isolate and activate the deep core muscles without direct professional guidance. Furthermore, these exercises are typically confined to active training sessions and are not designed to provide continuous, passive stimulation. At present, there is no known technique or device that activates the body's deep core muscles by leveraging the myofascial and neurological connections originating from the oral cavity, particularly from the tongue.

### SUMMARY OF THE INVENTION

In view of the above mentioned, an object of the invention is to provide a tongue depression auxiliary device that activates the deep core muscles of the human body. This is achieved by applying a specific pressure to the tongue, which leverages myofascial connections and triggers a neuro-modulating mechanism, effective whether the user is conscious or not.

In order to achieve the aforementioned object, the invention discloses a tongue depression auxiliary device configured for use in an oral cavity having an upper row of teeth, a lower row of teeth, and a tongue, the tongue depression auxiliary device comprising: a brace, configured to be detachably sleeved in the oral cavity, wherein the brace comprises a denture and a socket, and the socket is located on the denture; and a neuro-modulating member, connected to the brace, wherein a bottom side of the neuro-modulating member comprises at least one protrusion configured to exert a concentrated pressure on a specific functional region of the tongue, the concentrated pressure being configured to sufficiently stimulate a deep myofascial layer of the tongue that contributes to an activation of a deep core muscle, wherein the concentrated pressure is proportional to a length of the protrusion of the neuro-modulating member protruded from the bottom side of the brace.

In summary, the tongue depression auxiliary device of the invention has the following advantages:
(1) Direct Core Muscle Activation: By exerting a concentrated pressure on a specific functional region of the tongue, the device stimulates the deep myofascial layer, effectively engaging the body's entire core stability chain (e.g., the diaphragm, transversus abdominis). This provides a direct and passive method for activating muscles that are often difficult to engage consciously.
(2) Enhanced Neuromuscular Control: The device triggers a neuro-modulating mechanism by stimulating critical nerves in the tongue, such as the glossopharyngeal nerve. This enhances the user's proprioceptive feedback and improves the central nervous system's ability to control posture and balance.
(3) Improved Physiological Performance: As a result of better core stability and optimized neuromuscular function, the device helps to improve muscle power output, strengthen overall physical fitness, and promote more efficient breathing patterns, such as abdominal breathing. This can also lead to stress relief and an improvement in conditions like snoring or insomnia.
(4) Broad Applicability: The device is effective whether the user is awake, performing daily activities, exercising, or even sleeping. Its simple, non-invasive design allows for continuous and long-term use, helping to train and reshape the user's neuromuscular habits over time.

In order to enable the examiner to have a further understanding and recognition of the technical features of the invention and the technical efficacies that can be achieved, preferred embodiments in conjunction with detailed explanation are provided as follows.

### BRIEF DESCRIPTION OF THE DRAWINGS

FIG. 1 is a perspective view of a tongue depression auxiliary device of the invention.

FIG. 2 is a rear view of the tongue depression auxiliary device of the invention.

FIG. 3 is a cross-sectional side view of the tongue depression auxiliary device of the invention being worn in an oral cavity.

FIG. 4 is a cross-sectional side view of a general oral cavity wearing the tongue depression auxiliary device with a T-shape neuro-modulating member of the invention.

### DETAILED DESCRIPTION OF THE INVENTION

In order to understand the technical features, content and advantages of the invention and its achievable efficacies, the invention is described below in detail in conjunction with the figures, and in the form of embodiments, the figures used herein are only for a purpose of schematically supplementing the specification, and may not be true proportions and precise configurations after implementation of the invention; and therefore, relationship between the proportions and configurations of the attached figures should not be interpreted to limit the scope of the claims of the invention in actual implementation. In addition, in order to facilitate understanding, the same elements in the following embodiments are indicated by the same referenced numbers. And the size and proportions of the components shown in the drawings are for the purpose of explaining the components and their structures only and are not intending to be limiting.

Unless otherwise noted, all terms used in the whole descriptions and claims shall have their common meaning in the related field in the descriptions disclosed herein and in other special descriptions. Some terms used to describe in the present invention will be defined below or in other parts of the descriptions as an extra guidance for those skilled in the art to understand the descriptions of the present invention.

The terms such as “first”, “second”, “third” used in the descriptions are not indicating an order or sequence, and are not intending to limit the scope of the present invention. They are used only for differentiation of components or operations described by the same terms.

Moreover, the terms “comprising”, “including”, “having”, and “with” used in the descriptions are all open terms and have the meaning of “comprising but not limited to”.

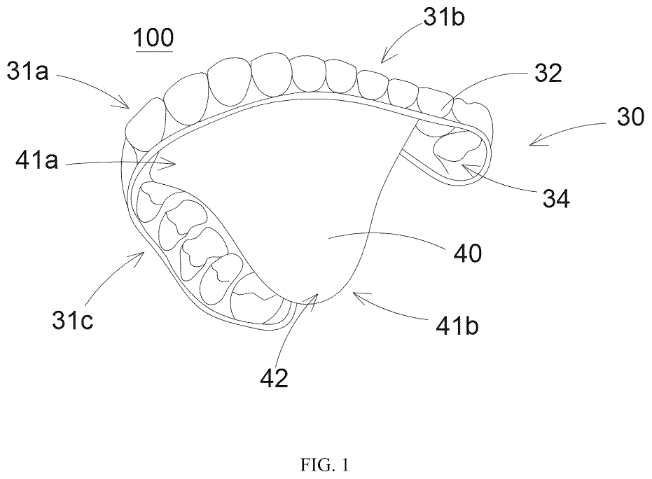

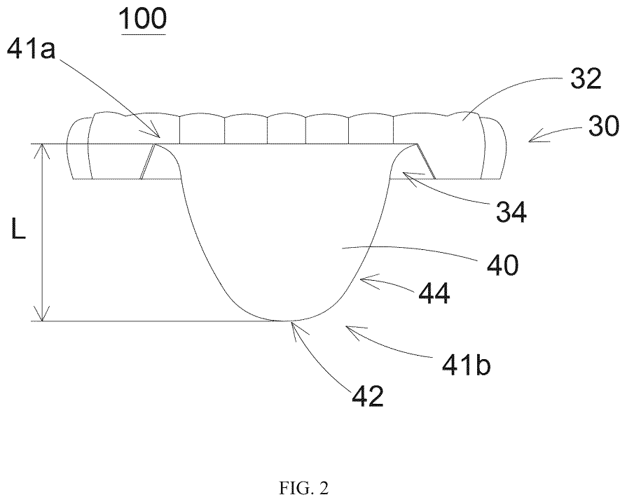

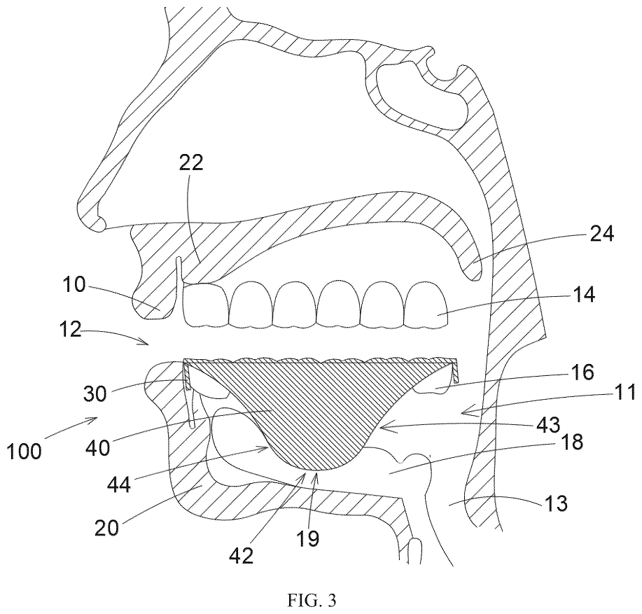

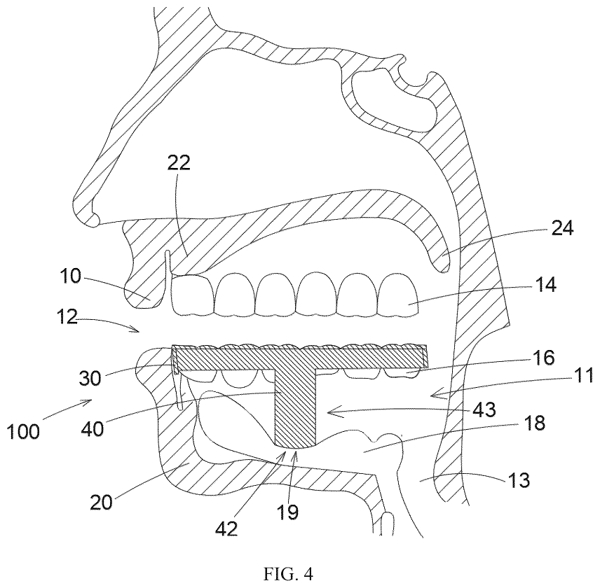

The core technical principle of the present invention is based on the discovery that applying a specific, concentrated pressure to a key functional region of the tongue can serve as a trigger to activate the deep core muscles of the body. This activation is achieved through two interconnected pathways: a myofascial pathway and a neurological pathway. From a myofascial perspective, the neuro-modulating member of the tongue depression auxiliary device exerts a concentrated pressure via its protrusion onto the deep myofascial layer of the tongue. This stimulation engages a biomechanical chain described as the “Deep Front Line,” which links the tongue to the hyoid muscles and the diaphragm. Concurrently, from a neurological perspective, this same pressure stimulates critical cranial nerves in the tongue, such as the glossopharyngeal nerve, triggering a synergistic neuro-modulating mechanism. The combination of these stimuli results in the enhanced activation of deep core muscles, such as the diaphragm, transversus abdominis, pelvic floor, and multifidus, leading to improved postural control and optimized breathing patterns. The following detailed description, in conjunction with the accompanying figures, will elaborate on the specific structure of the tongue depression auxiliary device that enables the implementation of this principle. Please refer to FIG. 1 to FIG. 4. FIG. 1 is a perspective view of a tongue depression auxiliary device of the invention; FIG. 2 is a rear view of the tongue depression auxiliary device of the invention; FIG. 3 is a cross-sectional side view of the tongue depression auxiliary device of the invention being worn in an oral cavity; and FIG. 4 is a cross-sectional side view of a general oral cavity wearing the tongue depression auxiliary device with a T-shape neuro-modulating member of the invention. A tongue depression auxiliary device 100 of the invention is used in an oral cavity 10, wherein the oral cavity 10 comprises an oral cavity opening 12, an upper row of teeth 14, a lower row of teeth 16, a tongue 18, a lower jaw 20 on the lower row of teeth 16 close to a side of the oral cavity opening 12, and a uvula 24 connected to an upper jaw 22 away from the side of the oral cavity opening 12.

The tongue depression auxiliary device 100 of the invention is composed of a brace 30 and a neuro-modulating member 40. Materials of the brace 30 and the neuro-modulating member 40 are such as but not limited to rubber, resin or plastic materials. The neuro-modulating member 40 can be made of rigid material to force the tongue 18 toward the lower jaw 20 so as to ensure a consistent and targeted transmission of the concentrated pressure to the deep myofascial layer without deformation. Alternatively, the neuro-modulating member 40 can be made of elastic material to force the tongue 18 toward the lower jaw 20 so as to provide a responsive counter-force to the tongue's movement, thereby enhancing a user's proprioceptive feedback and contributing to the neuro-modulating effect. Materials of the brace 30 and the neuro-modulating member 40 can be the same or different, and a hardness can also be the same or different. The brace 30 is detachably sleeved in the oral cavity 10, and the neuro-modulating member 40 is connected to the brace 30. The brace 30 is, for example, detachably sleeved on the upper row of teeth 14. In the invention, it is a preferred example that the brace 30 is sleeved on the lower row of teeth 16, but the invention is not limited thereto. The brace 30 can also be optionally and detachably sleeved on the upper row of teeth 14, or is sleeved on the upper row of teeth 14 and the lower row of teeth 16 at the same time. A peripheral edge of a top side 41a of the neuro-modulating member 40 is, for example, integrally connected with the brace 30, and the neuro-modulating member 40 can also be, for example, detachably connected with the brace 30. The neuro-modulating member 40 extends from the brace 30 toward the lower jaw 20 to contact with and forcibly push the tongue 18. The external contour and internal structure of the neuro-modulating member 40 may vary. In a specific embodiment, the external contour is defined by a downwardly tapering and convex shape, as shown in the figures. In other embodiments, however, it may be configured as another geometric shape (e.g., columnar or conical) capable of effectively supporting and displacing the tongue 18. For example, the external contour can be defined by T-shape external contour. Separately, irrespective of the external contour, the internal structure can be a solid bump (as shown in FIG. 3), an open-ended hollow bump, or a close-ended hollow bump. The bottom surface of the neuro-modulating member, configured to contact the specific functional region 19 of the tongue 18, can be a flat surface, a curved surface, or another shape with either a smooth or a non-smooth finish. A bottom side 41b of the neuro-modulating member 40 comprises at least one protrusion 42 in contact with a specific functional region 19 of the tongue 18. The protrusion 42 is configured to exert a concentrated pressure on a specific functional region 19 of the tongue 18, wherein the concentrated pressure is configured to sufficiently stimulate a deep myofascial layer of the tongue 18 that contributes to an activation of a deep core muscle of the human body of the user. The number of protrusion 42 can be one or more than one. In other words, when the brace 30 of the invention is sleeved on the teeth, the neuro-modulating member 40 will forcibly push the tongue 18 toward the lower jaw 20, such as making the tongue 18 concave, or even making the tongue 18 (including tongue tip, tongue body and root of tongue) move forward in a direction of the oral cavity opening 12, that is, away from a throat 13. As long as the user wears the tongue depression auxiliary device, it stimulates the deep myofascial layer of the tongue 18, which in turn contributes to the activation of deep core muscles, regardless of whether the user is awake or asleep. The specific functional region 19 of the tongue 18 targeted by the neuro-modulating member 40 is preferably the posterior third of the tongue 18, overlying the underlying hyoid muscles. The specific functional region 19 is configured to be an area where the concentrated pressure stimulates the glossopharyngeal nerve and the hyoid muscle group to facilitate the neuro-modulating mechanism. This specific functional region 19 is critical as it is anatomically linked to the deep front line and is dense with nerve endings that contribute to the neuro-modulating mechanism. Pressure on this region is believed to most effectively stimulate the hyoid muscle group and trigger the neuromuscular cascade leading to deep core muscle activation.

As a direct result of activating the deep core muscles, particularly the diaphragm, via the neuro-modulating member 40, a user's breathing pattern is optimized. This optimization can lead to more efficient respiration and, consequently, may enhance physical endurance and improve conditions related to inefficient breathing patterns, such as snoring. This effect is a beneficial outcome of the claimed core muscle activation mechanism, rather than a result of mere mechanical expansion of the airway. In detail, a throat opening 11 is a breathing passage, and the throat opening 11 is located above the throat 13 and adjacent to the uvula 24 and the tongue 18 in the oral cavity 10, so the throat opening 11 can also be expanded naturally by pushing the tongue 18. In other words, a cross-sectional area of the throat opening 11 after the tongue 18 being forcibly moved is larger than a cross-sectional area of the throat opening 11 without the tongue 18 being pushed, which means that the breathing passage is smoother. The neuro-modulating member 40 not only presses the tongue 18, but the neuro-modulating member 40 of the invention further pushes the tongue 18 toward the lower jaw 20 to deform the tongue 18 and even to force the tongue 18 move downward/forward to expand an intake area (cross-sectional area)/an expiratory area of the throat opening 11, and also to stimulate a fascia to activate self-healing ability. The tongue depression auxiliary device 100 of the invention is capable of increasing an amount of air intake and ventilation, and also capable of reducing a frequency of inhalation and ventilation, which means that a vital capacity can be increased. When a user does exercise (especially vigorous exercise), it is not easy to feel tired, and increasing lung capacity will have a positive effect on human body functions such as the cardiovascular system, lymphatic system, and autonomic nervous system.

The bottom side 41b of the neuro-modulating member 40 protrudes from a bottom side of the brace 30 to form the protrusion 42, and the bottom side 41b of the neuro-modulating member 40 extends (protrudes) a length L from the bottom side of the brace 30 toward a direction of the lower jaw 20, the length L is, for example, any numerical value between about 1 cm and about 10 cm, so that the bottom side 41b of the neuro-modulating member 40 supports the tongue 18, and even the tongue 18 is pushed a distance toward the lower jaw 20. And, the protrusion 42 has a contact area, which is ranged from 0.25 cm² to 4 cm² and configured to exert the concentrated pressure sufficient to cause a release of adhesions within the deep myofascial layer of the tongue 18. As a result, the protrusion 42 is configured to exert the concentrated pressure on the specific functional region 19 of the tongue 18 in a manner sufficient to achieve a therapeutic release of adhesions within the deep myofascial layer. A distance by which the bottom side 41b of the neuro-modulating member 40 extends corresponds to a distance by which the tongue 18 is pushed. The greater a distance the tongue 18 is pushed, the greater an extent the throat opening 11 is expanded. Moreover, if the protrusion 42 of neuro-modulating member 40 is provided with a stretchable structure, the degree of the concentrated pressure applied by the protrusion 42 can be adjustable by modulating the value of the length L of the protrusion 42. In addition, the magnitude of the concentrated pressure is user-adjustable by altering the user's jaw position. Namely, the magnitude of the concentrated pressure can be adjusted by the user by varying the opening of their mouth. And, the concentrated pressure, which is configured to be exerted on the specific functional region of the tongue 18 by the protrusion 42, is proportional to a length L of the protrusion 42 of the neuro-modulating member protruded from the bottom side of the brace 30. Therefore, the tongue depression auxiliary device of the present invention allows a user to maintain the stimulation intensity within an effective therapeutic window, avoiding excessive pressure that could lead to a decrease in recovery efficiency or trigger defensive muscle tension. Moreover, the magnitude of the concentrated pressure is determined by its ability to produce the most effective mechanical stimulation on the targeted deep myofascial layer, without triggering a defensive neuromuscular response. Accordingly, this is a value that requires continuous calibration through biofeedback, such as the user's proprioceptive feedback, rather than being a standardized or pre-set parameter. And, the magnitude of the concentrated pressure can be adjusted by the user by opening his mouth. Nevertheless, the magnitude of the concentrated pressure can be ranged from 25 kPa to 2,400 kPa, preferably, from 200 kPa to 800 kPa.

A key innovation of the neuro-modulating member 40 of the present invention lies in its structure, which is capable of simultaneously delivering two different types of physical stimuli to the tongue 18. Specifically, the protrusion 42 of the neuro-modulating member 40 applies a concentrated pressure on the specific functional region 19 of the tongue, which is configured to release adhesions within the deep myofascial layer. Concurrently, a surrounding surface 44 of the neuro-modulating member surrounding the protrusion is configured to optionally apply a distributed pressure across a surrounding region of the tongue adjacent to the specific functional region 19. This distributed pressure helps to induce a relaxation response in the user's body. Crucially, the concentrated pressure exerted by the protrusion and the distributed pressure exerted by the surrounding surface are configured to produce a synergistic neuro-modulating effect. This synergy, combining deep tissue stimulation with broad area relaxation, serves as the primary driving source for achieving neuro-modulation and deep core muscle activation more effectively.

The top side 41a and a surface 41c of the neuro-modulating member 40 are preferably not protruding from (that is, flush with or lower than) a top side of the brace 30. Yet another feature of the invention is that the neuro-modulating member 40 only pushes the tongue 18, does not push the upper jaw 22 or the uvula 24, and does not need to abut against the upper jaw 22 or the uvula 24, so whether the brace 30 or the neuro-modulating member 40 will not hinder air from entering the throat opening 11, and therefore the invention does not need additional ventilation holes. For example, when using the tongue depression auxiliary device 100, the neuro-modulating member 40 only contacts the tongue 18 with the bottom side 41b, and the surface 41c of the top side 41a of the neuro-modulating member 40 does not contact with the upper jaw 22 or the uvula 24. That is to say, the top side 41a of the neuro-modulating member 40 is separated from the upper jaw 22 or the uvula 24 with a gap, and the surface 41c of the top side 41a is substantially not protruding from (that is, flush with or lower than) the top side of the brace 30. The surface 41c is preferably flat, inclined or concave to form a flow guide surface, and the surface 41c is more preferably flat so as not to affect a respiratory airflow and to be capable of guiding the airflow.

In addition, an outer contour of the neuro-modulating member 40 is downwardly convex, and can be symmetrical or asymmetrical, for example, in a conical convex shape, that is, a width (cross-sectional area) of the top side 41a of the neuro-modulating member 40 is larger than a width (cross-sectional area) of the bottom side 41b. Moreover, an outer contour of the neuro-modulating member 40 can be in any shape, as long as the bottom side 41b of the neuro-modulating member 40 is capable of substantially pushing the tongue 18, the neuro-modulating member 40 in any shape can be applied in the invention, and the surface 41c of the top side 41a of the neuro-modulating member 40 is preferably to have a guide surface capable of guiding airflow. Furthermore, the surface 41c of the top side 41a of the neuro-modulating member 40 can also be in other forms that help guide an airflow, not limited to a horizontal plane, an arcuate surface or an inclined plane, so as to avoid reducing respiratory airflow. The neuro-modulating member 40 can, for example, extend vertically from top to bottom in a direction from the oral cavity opening 12 toward the throat opening 11, or for example, incline from top to bottom in a direction from the oral cavity opening 12 toward the throat opening 11, that is, incline downwardly away from the oral cavity opening 12. Similarly, the neuro-modulating member 40 can also, for example, incline from top to bottom from an inner side toward an outer side of the oral cavity opening 12, that is, incline downwardly toward a direction close to the oral cavity opening 12. For example, a width (cross-sectional area) of the neuro-modulating member 40 gradually decreases from the top side 41a toward the bottom side 41b to form the protrusion 42 with a drainage space 43. Therefore, when the neuro-modulating member 40 of the invention abuts against the tongue 18 with the bottom side 41b, saliva in the oral cavity 10 can automatically flow into the throat 13 for swallowing through the drainage space 43 between the top side 41a and the bottom side 41b, so a phenomenon of accumulation of saliva that cannot be eliminated does not occur. Of course, there is no need to additionally dispose drainage holes on the neuro-modulating member 40, and naturally there will be no problem of dirt accumulated in the drainage holes. And, the cross-sectional area of the neuro-modulating member 40 is configured to concentrate a user-applied force onto the protrusion 42, thereby enhancing the stimulation of the deep myofascial layer of the tongue 18. The neuro-modulating member is configured to trigger a neuro-modulating mechanism that results in the activation of the deep core muscle by supporting and pushing the tongue into a specific functional posture, such as in a concave form, wherein said deep core muscle is selected from a group consisting of a diaphragm, a pelvic floor, a transversus abdominis, and a multifidus.

The brace 30 of the tongue depression auxiliary device 100 of the invention is, for example, a U-shaped structure, which roughly matches an outer shape of the upper jaw 22 and/or the lower jaw 20 of the user. The brace 30 has a front central portion 31a, a left side portion 31b, and a right side portion 31c. The brace 30 at least comprises a denture 32 and a socket 34, wherein the socket 34 is located on the denture 32. The socket 34 can be a U-shaped groove or optionally formed with an outer shape suitable for closely fitting the user's teeth. An opening direction of the socket 34 of the bracket 32 is towards the lower row of teeth 16 (and/or can also be towards the upper row of teeth 14) to be sleeved, so that the socket 34 can be used to sleeve the brace 30 on the lower row of teeth 16 and/or the upper row of teeth 14 of the user. In the invention, the brace 30 being sleeved on the lower row of teeth 16 is used as a preferred example, so an opening direction of the socket 34 is towards the lower row of teeth 16.

The tongue depression auxiliary device 100 of the invention is capable of forcibly pushing the tongue 18 in order to open the throat opening 11 in the oral cavity 10, so an amount of air intake and oxygen intake per unit time will also increase accordingly, and therefore is capable of effectively optimizing physical fitness and increasing muscle power output. A greater extent the throat opening 11 is stretched open, the better the above-mentioned effects. Since the tongue depression auxiliary device 100 of the invention forces the tongue 18 to displace toward the lower jaw 20, it can not only exercise and activate the fascia of the tongue 18 after wearing the tongue depression auxiliary device 100 for a period of time (for example, several days), but can also train the tongue 18 to get used to a pushed state. The invention is capable of changing the user's breathing habits from oral breathing to nasal breathing. In addition, the invention is also capable of effectively training the user to get used to breathing by abdominal breathing method. In other words, the invention enables the user to completely or partially change the original thoracic breathing (TB) to abdominal breathing (AB). A gas exchange rate in the alveoli that can be carried out by abdominal breathing is higher, and inspiratory volume/ventilation volume will increase, thereby reducing a number and a frequency of breathing. Taking a normal adult's breathing rate of about 12 to 16 times per minute as an example, the tongue depression auxiliary device 100 of the invention is capable of reducing the user's breathing times per minute. It can be known that, no matter at any time, such as reading, exercising or sleeping, especially during vigorous exercise, as long as the user wears the tongue depression auxiliary device 100 in the oral cavity 10, vital capacity can be increased and inhalation/ventilation frequencies can be reduced. Naturally, it is not easy to feel tired and the user is capable of maintaining calmness and relieving stress, it will be more efficient in doing anything with better effects. Furthermore, one of the additional effects of the invention is that if the tongue depression auxiliary device 100 is worn during sleep, a cross-sectional area of the throat opening 11 will not reduce due to relaxation and retreat of the muscles of the tongue 18, so snoring can be avoided and sleep apnea can be solved. In addition, since the tongue depression auxiliary device 100 of the invention is capable of forcibly pushing the tongue 18 to open the throat opening 11 in the oral cavity 10, instead of just abutting against the tongue 18 to prevent the tongue 18 from retreating (retracting), an amount of air intake and oxygen intake per unit time will also increase accordingly, by which the user can be trained to increase lung capacity, effects are more obvious especially during exercise (especially during vigorous exercise or long-term exercise), so the tongue depression auxiliary device 100 is capable of effectively optimizing physical fitness and improving muscle power output. When the tongue 18 is pushed, because the tongue 18 as a whole is pulled, even the root of the tongue 18 or even the throat 13 will be pulled, so the corresponding fascia will be stimulated and activated, which can help the user to exert his or her own self-healing ability.

A key technology of the present invention is the demonstration that the application of a concentrated pressure to a specific functional area of the tongue, via a neuro-modulating member, can serve as a “hidden switch” for the activation of a deep core muscle, including the diaphragm, pelvic floor, transversus abdominis, and multifidus. This is achieved through several interconnected mechanisms. Firstly, from a myofascial perspective, the tongue is not an isolated organ but rather forms a tight mechanical chain with the mandible, cervical spine, and diaphragm via the hyoid muscles, as described in the “Deep Front Line” theory. Applying force via the protrusion of the neuro-modulating member activates this entire linkage, which stabilizes the craniocervical posture and enhances core efficiency. Secondly, the device optimizes breathing patterns by guiding the tongue into a specific functional posture. This promotes efficient nasal breathing, increases diaphragm utilization, and facilitates the synchronous activation of the transversus abdominis and diaphragm for improved intra-abdominal pressure control. Thirdly, it triggers a neuro-modulating mechanism. Applying concentrated pressure to the densely innervated tongue stimulates cranial nerves (such as the glossopharyngeal nerve, CN V, and CN XII), which, through integration by the Central Nervous System, enhances core muscle tension and postural control.

Based on these principles, the device of the present invention achieves several specific technical benefits. It directly promotes the activation of the deep core muscle by having the neuro-modulating member exert a concentrated pressure sufficient to stimulate the deep myofascial layer of the tongue, effectively engaging the body's entire core stability chain. This leads to improved breathing and core pressure management, as guiding the tongue into a specific functional posture fosters a nasal breathing pattern and increases the synchronous tension of the diaphragm and transversus abdominis. Consequently, the device enhances overall postural control and balance; by stabilizing the head and neck, it cohesively influences the pelvis and lower limbs, thereby enhancing the user's proprioceptive feedback and postural adjustment capabilities, while also achieving a therapeutic release of adhesions within the deep myofascial layer. These combined effects can significantly boost athletic performance and rehabilitation efficiency.

In summary, the tongue depression auxiliary device of the invention has the following advantages:
(1) Direct Core Muscle Activation: By exerting a concentrated pressure on a specific functional region of the tongue, the device stimulates the deep myofascial layer, effectively engaging the body's entire core stability chain (e.g., the diaphragm, transversus abdominis). This provides a direct and passive method for activating muscles that are often difficult to engage consciously.
(2) Enhanced Neuromuscular Control: The device triggers a neuro-modulating mechanism by stimulating critical nerves in the tongue, such as the glossopharyngeal nerve. This enhances the user's proprioceptive feedback and improves the central nervous system's ability to control posture and balance.
(3) Improved Physiological Performance: As a result of better core stability and optimized neuromuscular function, the device helps to improve muscle power output, strengthen overall physical fitness, and promote more efficient breathing patterns, such as abdominal breathing. This can also lead to stress relief and an improvement in conditions like snoring or insomnia.
(4) Broad Applicability: The device is effective whether the user is awake, performing daily activities, exercising, or even sleeping. Its simple, non-invasive design allows for continuous and long-term use, helping to train and reshape the user's neuromuscular habits over time.

Note that the specification relating to the above embodiments should be construed as exemplary rather than as limitative of the present invention, with many variations and modifications being readily attainable by a person of average skill in the art without departing from the spirit or scope thereof as defined by the appended claims and their legal equivalents.

What is claimed is:

1. A tongue depression auxiliary device configured for use in an oral cavity having an upper row of teeth, a lower row of teeth, and a tongue, the tongue depression auxiliary device comprising:
a brace, configured to be detachably sleeved in the oral cavity, wherein the brace comprises a denture and a socket, and the socket is located on the denture; and
a neuro-modulating member, connected to the brace, wherein a bottom side of the neuro-modulating member comprises at least one protrusion configured to exert a concentrated pressure on a specific functional region of the tongue, the concentrated pressure being configured to stimulate a deep myofascial layer of the tongue that contributes to an activation of a deep core muscle, wherein the concentrated pressure is proportional to a length of the protrusion of the neuro-modulating member protruded from the bottom side of the brace.

2. The tongue depression auxiliary device as claimed in claim 1, wherein a peripheral edge of a top side of the neuro-modulating member is connected with the brace, and the bottom side of the neuro-modulating member protrudes the length from the bottom side of the brace to form the protrusion, which is configured to exert the concentrated pressure on the specific functional region of the tongue in a manner sufficient to achieve a therapeutic release of adhesions within the deep myofascial layer.

3. The tongue depression auxiliary device as claimed in claim 1, wherein a cross-sectional area of the neuro-modulating member decreases gradually from a top side toward the bottom side to form the protrusion with a drainage space.

4. The tongue depression auxiliary device as claimed in claim 3, wherein the protrusion of the neuro-modulating member protrudes the length from the bottom side of the brace, and the length of the protrusion is ranged from 1 cm to 10 cm, and the protrusion has a contact area, which is ranged from 0.25 cm² to 4 cm² and configured to exert the concentrated pressure sufficient to cause a release of adhesions within the deep myofascial layer of the tongue, wherein the concentrated pressure is ranged from 25 kPa to 2,400 kPa.

5. The tongue depression auxiliary device as claimed in claim 1, wherein a cross-sectional area of the neuro-modulating member decreases gradually from a top side toward the bottom side to form the protrusion, and the cross-sectional area is configured to concentrate a user-applied force onto the protrusion, thereby enhancing the stimulation of the deep myofascial layer.

6. The tongue depression auxiliary device as claimed in claim 1, wherein the neuro-modulating member is configured to trigger a neuro-modulating mechanism that results in the activation of the deep core muscle by supporting and pushing the tongue into a specific functional posture, wherein said deep core muscle is selected from a group consisting of a diaphragm, a pelvic floor, a transversus abdominis, and a multifidus.

7. The device as claimed in claim 1, wherein the specific functional region is configured to be an area of the tongue overlying the hyoid muscles.

8. The device as claimed in claim 1, wherein the specific functional region is configured to be an area where the concentrated pressure stimulates the glossopharyngeal nerve and the hyoid muscle group to facilitate the neuro-modulating mechanism.

9. The tongue depression auxiliary device as claimed in claim 1, wherein the brace is configured to be detachably sleeved on the lower row of teeth and/or the upper row of teeth.

10. The tongue depression auxiliary device as claimed in claim 1, wherein the neuro-modulating member is defined by a T-shape external contour or a downwardly tapering and convex external contour.

11. The tongue depression auxiliary device as claimed in claim 1, wherein the bottom side of the neuro-modulating member is configured to be in contact with the specific functional region of the tongue, which is an area of the tongue overlying the hyoid muscle, and a top side of the neuro-modulating member is configured to be separated from an upper jaw of the oral cavity with a gap.

12. The tongue depression auxiliary device as claimed in claim 1, wherein the brace is configured to be detachably sleeved on the lower row of teeth, and a surface of a top side of the neuro-modulating member is flat to be a flow guide surface.

13. The tongue depression auxiliary device as claimed in claim 1, wherein the neuro-modulating member, which is configured to stimulate the deep myofascial layer of the tongue, is made of rubber, resin or plastic material.

14. The tongue depression auxiliary device as claimed in claim 1, wherein the neuro-modulating member is made of rigid material and is configured to ensure a consistent and targeted transmission of the concentrated pressure to the deep myofascial layer without deformation.

15. The tongue depression auxiliary device as claimed in claim 1, wherein the neuro-modulating member is made of elastic material and is configured to provide a responsive counter-force to the tongue's movement, thereby enhancing a user's proprioceptive feedback and contributing to the neuro-modulating effect.

16. The tongue depression auxiliary device as claimed in claim 1, wherein the number of protrusion is more than one.

17. The tongue depression auxiliary device as claimed in claim 1, wherein the neuro-modulating member is an open-ended hollow bump, a close-ended hollow bump, or a solid bump with a smooth outside surface.

18. The tongue depression auxiliary device as claimed in claim 1, wherein the length of the protrusion is adjustable to thereby change the degree of the concentrated pressure configured to be exerted on the tongue.

19. The tongue depression auxiliary device as claimed in claim 1, wherein an outer contour of the neuro-modulating member is downwardly convex and symmetrical with a drainage space.

20. The tongue depression auxiliary device as claimed in claim 1, wherein a surrounding surface of the neuro-modulating member surrounding the protrusion is configured to apply a distributed pressure across a surrounding region of the tongue adjacent to the specific functional region, and wherein the concentrated pressure exerted by the protrusion and the distributed pressure exerted by the surrounding surface are configured to produce a synergistic neuro-modulating effect.
